(19) United States
(12) Patent Application Publication
(10) Pub. No.: US 2011/0086809 A1
(43) Pub. Date: Apr. 14, 2011

(54) Enteral Administration Of Arginine-Glutamine Dipeptide To Support Retinal, Intestinal, Or Nervous System Development

(76) Inventors: Joshua Anthony, Evansville, IN (US); Kristin Morris, Evansville, IN (US); Maria Grant, Fairfield, FL (US); Joseph Neu, Gainesville, FL (US)

(21) Appl. No.: 12/578,922

(22) Filed: Oct. 14, 2009

(51) Int. Cl. A61K 38/05; A61P 3/02; A61K 47/00

(52) U.S. Cl. 514/21.91; 424/439

(57) ABSTRACT

A method for supporting retinal, intestinal, and/or nervous system development in a neonate is provided. The method involves enterally administering arginine-glutamine dipeptide to a neonate.

### TECHNICAL FIELD

The present disclosure relates to a method generally comprising enterally administering arginine-glutamine dipeptide in an amount that is effective to support retinal, intestinal, or nervous system development.

### SUMMARY OF THE DISCLOSURE

Briefly, one embodiment of the present disclosure relates to a novel method for supporting retinal, intestinal, and/or nervous system development in a neonate. The method generally includes enterally administering arginine-glutamine dipeptide to a neonate to provide an amount of arginine-glutamine dipeptide that is effective to support retinal, intestinal, and/or nervous system development, especially in an infant not otherwise receiving nutrition adequate in arginine and glutamine.

In another embodiment, the present disclosure presents a novel infant formula. The infant formula generally includes arginine-glutamine dipeptide in an amount that is effective to support one or more of retinal, intestinal, and nervous system development in a neonate.

In yet another embodiment, the present disclosure is a human milk fortifier. The human milk fortifier generally includes arginine-glutamine dipeptide in an amount that is effective to support one or more of retinal, intestinal, and nervous system development in a neonate.

In a different embodiment, the present disclosure is an infant nutritional supplement. The infant nutritional supplement generally includes arginine-glutamine dipeptide in an amount that is effective to support one or more of retinal, intestinal, and nervous system development in a neonate.

Among the several embodiments of the present disclosure, therefore, may be the provision of compositions and practicing of methods which support retinal, intestinal, and/or nervous system development, and the provision of compositions and practicing of methods that treat hyperoxia symptoms, prevent retinopathy of prematurity (ROP), and/or reduce the risk of developing necrotizing enterocolitis (NEC).

These and other embodiments of the disclosure will be understood and become apparent upon review of the specification by those having ordinary skill in the art.

The present disclosure may be better understood by reference to the description that follows. It is to be understood that the disclosure is not limited in its application to the specific details as set forth in the following description. The disclosure is capable of other embodiments and of being practiced or carried out in various ways.

### BRIEF DESCRIPTION OF THE DRAWINGS

For a more complete understanding of the present disclosure, reference is now made to the following descriptions taken in conjunction with the accompanying drawings.

FIG. 1 is a bar chart showing the effect of enterally administering arginine-glutamine dipeptide in reducing the average nuclei per section of the eyes of neonatal mice exposed to hyperoxia to induce retinal angiogenesis at dosage levels of: zero (control); 1 g/kg·day; 2.5 g/kg·day; and 5 g/kg·day.

FIG. 2 is a bar chart quantifying the level of intestinal injury of neonatal mice under three different conditions: control; hyperoxic conditions; and hyperoxic conditions combined with arginine-glutamine dipeptide treatment.

FIG. 3 is a bar chart showing the effect of administering arginine-glutamine dipeptide in increasing Bcl-2 expression in the brain of neonatal mice exposed to hyperoxic conditions.

FIG. 4 is a bar chart showing the effect of administering arginine-glutamine dipeptide in decreasing brain caspase-3 activity in the brain of neonatal mice exposed to hyperoxic conditions.

### DETAILED DESCRIPTION OF THE PREFERRED EMBODIMENTS

The present disclosure provides compositions containing arginine-glutamine dipeptides and methods for administering the same. In particular, the subject disclosure provides enteral administration of arginine-glutamine dipeptide to support retinal, intestinal, and/or nervous system development. As used herein, “enteral” means through or within the gastrointestinal, or digestive, tract, and “enteral administration” includes oral feeding, intragastric feeding, transpyloric administration, or any other introduction into the digestive tract.

In a particularly effective embodiment, enteral administration of certain amounts of arginine-glutamine dipeptide is effective for facilitating healthy brain development, preventing retinopathy of prematurity (ROP), decreasing intestinal damage, and/or reducing the risk of developing necrotizing enterocolitis (NEC). ROP is a retinal blood vessel development disorder in premature infants. NEC is a leading cause of mortality and morbidity in neonatal intensive care units.

A dipeptide is a molecule consisting of two amino acids joined by a single peptide bond. In the present disclosure, the two amino acids utilized are arginine and glutamine.

The subject disclosure contemplates the enteral administration of arginine-glutamine dipeptide in any form that can be ingested and absorbed. Arginine and glutamine are contemplated as useful in the present disclosure as: (1) free amino acids or salts; (2) precursors of such amino acids; or (3) prodrugs. Whereas most amino acids exist bound together in various proteins, free amino acids are unbound. Amino acid precursors are substances from which the amino acids are formed. A prodrug may be based on a particular amino acid and is a pharmacological substance administered in an inactive (or significantly less active) form. Once administered, the prodrug is metabolized in vivo into the active compound.

In addition, an arginine-glutamine dipeptide itself may be administered. On the other hand, oligopeptides, peptides, proteins, protein hydrolysates, and any other materials that could serve as a source of the arginine-glutamine dipeptide may also be administered either as a substitute or an additive. Examples of such sources include peptides of polyarginine and polyglutamine, peptides containing blocks of polyarginine and polyglutamine, and peptides of alternating arginine and glutamine.

In the case of oligopeptides, peptides, and proteins that contain the arginine-glutamine dipeptide, these prodrug formulations may be designed with, for example, cleavage sites adjacent to each side of the arginine-glutamine dipeptide so that the dipeptide is generated upon exposure to enzymes, acids, or other factors.

In one embodiment, a polypeptide can be prepared with multiple arginine-glutamine dipeptides separated by cleavage sites. When the polypeptide is exposed to a cleaving factor, which breaks apart the polypeptide, it is separated into multiple arginine-glutamine dipeptides. This cleaving to create the dipeptide can be performed as part of a production process or in vivo as the result of, for example, digestive enzymes and/or acids.

As contemplated in the subject disclosure, where the arginine-glutamine dipeptide is provided by a prodrug, the prodrug can be converted to a biologically active compound at a controlled rate via passive (such as by aqueous hydrolysis) mechanisms or biologically-mediated (such as biocatalytic or enzymatic) mechanisms. An advantage of the in vivo conversion of the prodrug is that the ensuing dipeptide provides localized therapeutic effects in target disease tissue with high therapeutic margins of safety.

In one embodiment, the arginine-glutamine dipeptide provides the two amino acids in a dipeptide having improved water solubility, stability to sterilization, long-term stability, and bioavailability for humans and animals. An advantage of the dipeptide is its increased stability over free glutamine, for example, resulting in much lower cyclisation of glutamine into undesirable pyro-glutamate. In one form, the arginine-glutamine dipeptide of the present disclosure has an N-terminal amino acid, which is arginine, and a C-terminal amino acid, which is glutamine.

The arginine-glutamine dipeptide of the subject disclosure can be readily synthesized and/or formulated by a person skilled in the art having the benefit of the present disclosure. Alternatively, the dipeptides can be purchased commercially from, for example, Bachem Biosciences, Inc., which sells an arginine-glutamine dipeptide salt. The arginine-glutamine dipeptides can be of any purity or grade, and can be of a purity and grade that is suitable for inclusion in the diet of human infants.

The present disclosure is useful to support retinal, intestinal, and/or nervous system development in a neonate. The term “neonate” as used herein means a newborn infant, including premature infants, postmature infants, and full term newborns. Premature infants are babies born before 37 weeks of gestation. Postmature infants are babies born 2 weeks or more after 37 weeks of gestation. Full-term infants are born within 37 to 39 weeks of gestation. Typically, neonates are infants less than one month old.

In an embodiment of the disclosure, the neonate may be in need of support for retinal, intestinal, and/or nervous system development. A neonate may be in need of support for retinal, intestinal, and/or nervous system development, for example, after being placed in hyperoxic conditions. Hyperoxic conditions have higher than normal oxygen saturation levels. Premature infants are routinely placed in a hyperoxic environment for purposes of resuscitation and treatment of respiratory maladies.

Apoptotic neurodegeneration, abnormal vascularization, NEC, or other neurological deficits or infections may also be used to identify a neonate that may be in need of support for retinal, intestinal, and/or nervous system development. A neonate is typically in need of such support if it exhibits, or is at risk for developing, such pathologies. Premature infants and infants exposed to hyperoxic environments or other conditions that are known to increase the risk of neurological deficits or NEC are often in need of support for retinal, intestinal, and/or nervous system development.

In another embodiment of the disclosure, the arginine-glutamine dipeptide described herein may be used to prevent neovascularization in the retinal vascular endothelium, thus supporting healthy retinal development. Because retinal development is believed to be correlative to cognitive development, this may also support normal cognitive functions in the neonate.

In yet another embodiment, the arginine-glutamine dipeptides described herein may increase the expression of the protein Bcl-2 in the brain. The protein Bcl-2 is an anti-apoptotic protein that resides in the outer mitochondrial membrane and the membrane of the endoplasmic reticulum. High levels of the Bcl-2 protein protect cells from early death by apoptosis (cell death). The Bcl-2 protein can suppress apoptosis by preventing the activation of the caspases that carry out the process.

Thus, in an embodiment of the disclosure, the arginine-glutamine dipeptides described herein can decrease the level of caspases in the brain. Caspases are protease enzymes that play essential roles in apoptosis and inflammation. Certain caspases cleave other protein substrates within cells, resulting in the apoptotic process. Decreased levels of caspase may promote nervous system development.

In accordance with the teachings provided herein, aqueous compositions can be prepared that contain at least one arginine-glutamine dipeptide. The dipeptide can be added to enteral formulations, which can include nutritional supplements. As discussed in more detail below, in addition to the arginine-glutamine dipeptides of the subject disclosure, the formulas, supplements, or nutritional solutions can contain, for example, carbohydrates, lipids, fats, stabilizers, amino acids, peptides (including dipeptides, oligopeptides, and/or polypeptides), and/or proteins, vitamins, minerals and trace elements. The selection of the particular arginine-glutamine dipeptide formulation depends upon the particular use for the formulation. The administration of the arginine-glutamine dipeptide, rather than administration of free amino acids, permits administration of the same amount of amino acid residue in solutions, which are less hypertonic and, therefore, of lower osmolality.

In the present disclosure, the arginine-glutamine dipeptide may be enterally administered to a neonate. In an embodiment of the disclosure, the arginine-glutamine dipeptides described herein may reduce intestinal injury associated with enteral administration, thus protecting the structural integrity of the intestine. As used herein, “intestinal injury” includes inflammation, cellular damage, or any other event that compromises the physiological integrity of the intestine.

Enteral nutrition is safer and less expensive than total parenteral nutrition, which is the practice of feeding a person intravenously. Enteral administration is the preferred route for maintaining the integrity of the gastrointestinal tract. When possible, oral administration is desirable, because it is the normal method for infant nutrition and one that is understood and accepted by the infant and persons providing nutrition to the infant.

Enteral administration of the present arginine-glutamine dipeptide can be by any recognized method and can take place at any time. Formulations containing the active agents can be given once a day or multiple times per day. Administration of formulations containing the present arginine-glutamine dipeptide can be alternated with administration of formulations that do not contain the present arginine-glutamine dipeptide, or formulations that contain the present dipeptide at levels other than those that will provide arginine-glutamine dipeptide to the infant in a total amount of from about 100 mg/kg·day to about 1000 mg/kg·day.

The arginine-glutamine dipeptide of the present disclosure can be enterally administered to a neonate in any known and accepted form or manner. An embodiment of the present disclosure is a composition that can be an infant formula, human milk fortifier, or a nutritional or dietary supplement, and combinations thereof.

The infant formula, human milk fortifier, or nutritional supplement of the present disclosure can be milk-based, soy-based, based on other food sources, or combinations thereof. The composition may be prepared as a powder and/or a liquid for formulas prepared for infant populations. If in powder form or concentrate liquid form, the formula is diluted to normal strength with water to be in a form ready to consume. The inventive composition may be prepared as a nutritionally complete diet by including necessary nutrients, including vitamins and minerals at acceptable levels. The subject composition can be in the form of a dietary product such as an infant formula, milk substitute, and/or meal replacement or supplement.

One embodiment of the disclosure is a nutritional or dietary supplement that contains the arginine-glutamine dipeptide or a precursor thereof. The dietary supplement is designed to be administered along with a food or nutritional composition, such as infant formula, and can either be intermixed with the food or nutritional composition prior to ingestion by the subject, or can be administered to the subject either before or after ingestion of a food or nutritional composition. The subject dietary supplement contains an amount of arginine-glutamine dipeptide, or a precursor thereof, that is effective for the support of retinal, intestinal, and/or nervous system development.

The amount of the arginine-glutamine dipeptide or its salt or prodrug that is an effective amount is an amount sufficient to evoke the desired physiological response. This is generally an amount sufficient to produce lessening of one or more of the effects of apoptotic neurodegeneration or neurological morbidity, reduce the risk of developing NEC, positively influence normal retinal vascularization and/or have a prohibitive effect on the risk of developing ROP. In the case of apoptotic neurodegeneration, it is an amount sufficient to reduce neurological morbidity and/or to support normal cognitive development and motor outcomes in premature infants and/or other infants otherwise susceptible to neurological deficits in the immediate postnatal period.

In an embodiment of the present disclosure, the arginine-glutamine dipeptide is administered to a neonate in an amount that may be from about 0.001 to about 10,000 mg/kg·day (where the units of mg/kg·day refer to mg of the arginine-glutamine dipeptide per kg of infant body weight per day). The effective amount can also be from about 100 mg/kg·day to about 1000 mg/kg·day, or from about 200 mg/kg·day to about 800 mg/kg·day, or from about 250 mg/kg·day to about 600 mg/kg·day, or from about 300 mg/kg·day to about 600 mg/kg·day, or from about 300 mg/kg·day to about 500 mg/kg·day, or in an amount of about 500 mg/kg·day. Here, the amount of the active ingredients by weight refers to the amount of the equimolar combination of the arginine-glutamine dipeptide, or the amount of their salts or precursors sufficient to provide the stated amount of dipeptide.

In one embodiment, a novel infant formula containing the present arginine-glutamine dipeptide, or precursor thereof, is nutritionally complete. By the term “nutritionally complete” it is meant that the composition contains adequate nutrients to sustain healthy human life for extended periods. The infant formula of the disclosure contains ingredients which are designed to meet the nutritional needs of the human infant, namely a fat, protein, carbohydrate, and lipid source, a stabilizer, and other nutrients such as vitamins and minerals.

Besides the subject dipeptide, the composition of the disclosure can contain an additional nitrogen source (i.e., amino acids and/or protein) such that the total amount of amino acids or protein may be between about 1 g/100 kilocalories (kcal) to about 10 g/100 kcal of total composition, in some embodiments about 2 g/100 kcal to about 6 g/100 kcal. The amount of lipid source per 100 kcal of total composition may be greater than 0 g up to about 6 g, in some embodiments about 0.5 g to about 5.5 g, and in other embodiments about 2 g to about 5.5 g; and the amount of non-fiber carbohydrate source per 100 kcal of total composition may be about 5 g to about 20 g, and in some embodiments may be about 7.5 g to about 15 g.

As used herein, the subject infant formula is not meant to include natural milk, such as unmodified cow's milk, unmodified goat's milk, unmodified human milk, or any other unmodified natural product, but rather refers to a formulation made by man in whole or in part by intermixing two or more ingredients. Thus, the term “infant formula” means a composition that satisfies the nutrient requirements of an infant by being a substitute for human milk. In a particular embodiment, the infant formula, human milk fortifier or infant nutritional supplement is in a powdered form. In other embodiments, infant formula, human milk fortifier or infant nutritional supplement may be in a liquid or ready-to-use form.

In an embodiment, the infant formula for use in the present disclosure is nutritionally complete and contains suitable types and amounts of lipids, fats, carbohydrates, proteins, vitamins and minerals. The amount of lipid or fat typically can vary from about 3 to about 7 g/100 kcal. The amount of protein typically can vary from about 1 to about 5 g/100 kcal. The amount of carbohydrate typically can vary from about 8 to about 12 g/100 kcal.

When an additional protein source is included in the subject infant formula, it can be non-fat milk solids, a combination of non-fat milk solids and whey protein, a partial hydrolysate of non-fat milk and/or whey solids, soy protein isolates, or partially hydrolyzed soy protein isolates. The infant formula can be casein predominant or whey predominant.

The carbohydrate source in the infant formula can be any suitable carbohydrate known in the art to be suitable for use in infant formulas. Typical carbohydrate sources include sucrose, fructose, glucose, maltodextrin, lactose, corn syrup, corn syrup solids, rice syrup solids, rice starch, modified corn starch, modified tapioca starch, rice flour, soy flour, and the like.

The lipid source in the infant formula can be any lipid or fat known in the art to be suitable for use in infant formulas. Typical lipid sources include milk fat, safflower oil, egg yolk lipid, olive oil, coconut oil, palm oil, palm kernel oil, soybean oil, sunflower oil, fish oil and fractions derived thereof such as palm olein, medium chain triglycerides (MCT), and esters of fatty acids wherein the fatty acids are, for example, arachidonic acid, linoleic acid, palmitic acid, stearic acid, docosahexaenoic acid, eicosapentaenoic acid, linolenic acid, oleic acid, lauric acid, capric acid, caprylic acid, caproic acid, and the like. High oleic forms of various oils are also contemplated to be useful herein such as high oleic sunflower oil and high oleic safflower oil. MCT contains higher concentrations of caprylic and capric acid than typically found in conventional oils, e.g., approximately three-fourths of the total fatty acid content is caprylic acid and one-fourth is capric acid.

The infant formula of the disclosure also may contain emulsifiers and stabilizers such as soy lecithin, carrageenan, combinations thereof, and the like.

The infant formula of the disclosure may optionally contain other substances which may have a beneficial effect such as lactoferrin, nucleotides, nucleosides, immunoglobulins, combinations thereof, and the like.

The osmolality of the liquid infant formula of the disclosure (when ready to consume) may be between about 100 and 1100 mOsm/kg H₂O, in some embodiments, between about 200 and 700 mOsm/kg H₂O.

The infant formula of the disclosure can be sterilized, if desired, by techniques known in the art, for example, heat treatment such as autoclaving or retorting, and the like.

The infant formula of the disclosure can be packaged in any type of container known in the art to be used for storing nutritional products such as glass, lined paperboard, plastic, coated metal cans, and the like.

The infant formula of the disclosure should be shelf stable after reconstitution. By “shelf stable,” it is meant that the formula, in a form ready to consume, remains in a single homogenous phase (i.e., does not separate into more than one phase upon visual inspection) or that the thickener does not settle out as a sediment upon visual inspection after storage overnight in the refrigerator. With the thickened nature of the product, the formula of the disclosure also has the advantage of remaining fluid (i.e., does not gel into a solid mass when stored overnight in the refrigerator).

Conveniently, commercially available infant formula can be used. For example, Enfalac™, Enfamil®, Enfamil® Premature Formula, Enfamil® with Iron, Enfamil® LIPIL®, Lactofree®, Nutramigen®, Pregestimil®, and ProSobee® (available from Mead Johnson & Company, Evansville, Ind., U.S.A.) may be supplemented with suitable amounts of arginine-glutamine dipeptide and used in practice of the disclosure.

In some embodiments of the disclosure, the infant formula, human milk fortifier, or infant nutritional supplement contains additional components which may include probiotics, prebiotics, or additional long chain polyunsaturated fatty acids (LCPUFAs). The term “probiotic” means a microorganism that exerts beneficial effects on the health of the host. Any probiotic known in the art may be added, provided it is suitable for combination with the other components of the supplement. For example, the probiotic may be chosen from the group consisting of Lactobacillus and Bifidobacterium. Alternatively, the probiotic can be Lactobacillus rhamnosus GG.

In certain embodiments, the infant formula, human milk fortifier, or infant nutritional supplement of the present disclosure additionally comprises at least one prebiotic. The term “prebiotic”, as used herein, means a non-digestible food ingredient that stimulates the growth and/or activity of probiotics. In this embodiment, any prebiotic known in the art may be added, provided it is suitable for combination with the other components of the supplement. In a particular embodiment, the prebiotic can be selected from the group consisting of polydextrose, fructo-oligosaccharide, gluco-oligosaccharide, galacto-oligosaccharide, inulin, isomalto-oligosaccharide, xylo-oligosaccharide, lactulose, and combinations thereof.

In particular embodiments, the LCPUFA may be docosahexaenoic acid (DHA), arachidonic acid (ARA), and/or eicosapentaenoic acid (EPA). If used, the amount of ARA in the present disclosure may be from about 4 mg/100 kcal to about 100 mg/100 kcal. In another embodiment, the amount of ARA may be from about 10 mg/100 kcal to about 67 mg/100 kcal. In yet another embodiment, the amount of ARA may be from about 20 mg/100 kcal to about 50 mg/100 kcal. In a particular embodiment, the amount of ARA may be from about 25 mg/100 kcal to about 40 mg/100 kcal. In one embodiment, the amount of ARA is about 30 mg/100 kcal.

If administered as part of the present disclosure, the weight ratio of ARA:DHA may be from about 1:3 to about 9:1. In one embodiment of the present disclosure, this ratio is from about 1:2 to about 4:1. In yet another embodiment, the ratio is from about 2:3 to about 2:1. In one particular embodiment the ratio is about 2:1. In another particular embodiment of the disclosure, the ratio is about 1:1.5. In other embodiments, the ratio is about 1:1.3. In still other embodiments, the ratio is about 1:1.9. In a particular embodiment, the ratio is about 1.5:1. In a further embodiment, the ratio is about 1.47:1.

If administered as part of the present disclosure, the level of DHA may be between about 0.0% and 1.00% of fatty acids, by weight. In other embodiments, the level of DHA may be about 0.32% by weight. In some embodiments, the level of DHA may be about 0.33% by weight. In another embodiment, the level of DHA may be about 0.64% by weight. In another embodiment, the level of DHA may be about 0.67% by weight. In yet another embodiment, the level of DHA may be about 0.96% by weight. In a further embodiment, the level of DHA may be about 1.00% by weight.

If administered as part of the present disclosure, the level of ARA may be between 0.0% and 0.67% of fatty acids, by weight. In another embodiment, the level of ARA may be about 0.67% by weight. In another embodiment, the level of ARA may be about 0.5% by weight. In yet another embodiment, the level of DHA may be between about 0.47% and 0.48% by weight.

If administered as part of the present disclosure, the amount of DHA may be from about 2 mg/100 kcal to about 100 mg/100 kcal. In another embodiment, the amount of DHA may be from about 5 mg/100 kcal to about 75 mg/100 kcal. In yet another embodiment, the amount of DHA may be from about 15 mg/100 kcal to about 60 mg/100 kcal.

If administered as part of the present disclosure, the amount of ARA may be from about 4 mg/100 kcal to about 100 mg/100 kcal. In another embodiment, the amount of ARA may be from about 10 mg/100 kcal to about 67 mg/100 kcal. In yet another embodiment, the amount of ARA may be from about 20 mg/100 kcal to about 50 mg/100 kcal. In a particular embodiment, the amount of ARA may be from about 25 mg/100 kcal to about 40 mg/100 kcal. In one embodiment, the amount of ARA is about 30 mg/100 kcal.

If administered as part of the present disclosure, the effective amount of DHA may be from about 3 mg per kg of body weight per day to about 150 mg per kg of body weight per day. In one embodiment of the disclosure, the amount is from about 6 mg per kg of body weight per day to about 100 mg per kg of body weight per day. In another embodiment the amount is from about 15 mg per kg of body weight per day to about 60 mg per kg of body weight per day.

If administered as part of the present disclosure, the effective amount of ARA may be from about 5 mg per kg of body weight per day to about 150 mg per kg of body weight per day. In one embodiment of this disclosure, the amount varies from about 10 mg per kg of body weight per day to about 120 mg per kg of body weight per day. In another embodiment, the amount varies from about 15 mg per kg of body weight per day to about 90 mg per kg of body weight per day. In yet another embodiment, the amount varies from about 20 mg per kg of body weight per day to about 60 mg per kg of body weight per day.

The LCPUFA source may or may not contain EPA. In some embodiments, the LCPUFA used in the disclosure contains little or no EPA. For example, in certain embodiments the infant formulas used herein contain less than about 20 mg/100 kcal EPA; in some embodiments less than about 10 mg/100 kcal EPA; in other embodiments less than about 5 mg/100 kcal EPA; and in still other embodiments substantially no EPA.

If the composition of the disclosure is supplemented with oils containing LCPUFAs, it may be accomplished using standard techniques known in the art. For example, an equivalent amount of an oil which is normally present in a composition, such as high oleic sunflower oil, may be replaced with the LCPUFAs. If utilized, the source of the LCPUFAs can be any source known in the art such as marine oil, fish oil, single cell oil, egg yolk lipid, brain lipid, and the like. The LCPUFAs can be in natural form or refined form.

The disclosure provides a commercially acceptable product in terms of desired stability and physical characteristics and the product demonstrates little to no observable browning effect by-products associated with a Maillard reaction. Furthermore, the inventive composition is substantially homogeneous for an acceptable period after reconstitution (or for the shelf-life if prepared as a liquid). The disclosure may be particularly useful for infant formula preparations for the support of retinal, intestinal, and/or nervous system development, although it is equally applicable to other elemental diets specific to a selected population that is at risk of apoptotic neurodegeneration, ROP, and/or NEC.

Reference now will be made in detail to the embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment can be used on another embodiment to yield a still further embodiment. Thus, it is intended that the present disclosure cover such modifications and variations as come within the scope of the appended claims and their equivalents. Other objects, features and embodiments of the present disclosure are disclosed in or are obvious from the following detailed description. It is to be understood by one of ordinary skill in the art that the present discussion is a description of exemplary embodiments only, and is not intended as limiting the broader embodiments of the present disclosure.

### Example 1

This example illustrates the efficacy of enteral administration of arginine-glutamine dipeptide for the prevention of retinopathy of prematurity in a mouse model of oxygen-induced retinopathy.

All animals are treated in accordance with the ARVO “Statement for the Use of Animals in Ophthalmic and Vision Research.” Animal procedures were approved by the Institutional Animal Care and Use Committee of the University of Florida.

The C57BL6/J strain of timed pregnant mice was obtained from Jackson Laboratories (Bar Harbor, Me.). The mice were housed in the University of Florida Health Science Center Animal Care facilities. The pregnant mice gave birth to the mouse pups used in the experiments.

The overall purpose of the experiments in animals is to establish safety, appropriate dosage range, and efficacy prior to evaluation in human neonates at risk for retinopathy of prematurity. Since the ultimate purpose of the arginine-glutamine dipeptide is to provide a safe and easily absorbable preparation that can be used to provide appropriate nutritional intakes of arginine and glutamine in human infants who might not be receiving appropriate quantities of these amino acids in their diets, the dosages in the animal studies are modeled after human premature neonate recommended intakes.

In the neonatal mouse model of oxygen-induced retinopathy, 7-day old mice are placed with their nursing dams in a 75% oxygen atmosphere for 5 days. Mouse pups receive twice a day gavage feedings of arginine-glutamine dipeptide or control solution (50 μL) starting on postnatal day 12 (P12) and continuing through postnatal day 17 (P17). Gavage feeds include a control (0.9% sodium chloride) and the test compounds and different doses of arginine-glutamine dipeptide (1.0, 2.5 and 5 g/kg·day). The daily dosage of the dipeptide is divided evenly between the two daily gavage feedings.

After the fifth day following return to normoxia (P17), the animals are euthanized by injection of a lethal dose of a combination of ketamine (70 mg/kg body weight) and xylazine (15 mg/kg body weight) followed by cervical dislocation. The eyes are removed and fixed in 4% paraformaldehyde, embedded in paraffin, and sectioned. Pre-retinal nuclei are counted by masked observers. Efficacy of treatment is calculated as the percent average nuclei per section in the eyes of arginine-glutamine dipeptide treated animals versus control animals.

For total RNA isolations from retina, the animals are sacrificed and the eyes removed. The retina is then dissected away from the eye and stored in RNAlater® buffer (Ambion, Austin, Tex.) at 4° C. for subsequent isolation of protein or RNA.

Some of the eyes are taken for qualitative retinal flatmount analysis. For these mice, at the time of euthanasia, the mice are perfused with fluorescein isothiocyanate-labeled dextran to visualize the vasculature. The eyes are enucleated and incubated in 4% formaldehyde and then in phosphate buffered saline cell culture media. The neural retina is dissected from the retinal pigment epithelium-choroid-sclera complex and flatmounted with four to seven radial cuts and examined and photographed separately using confocal microscopy (with a MRC-1024 Confocal Laser Scanning System from Bio-Rad, Hercules, Calif.).

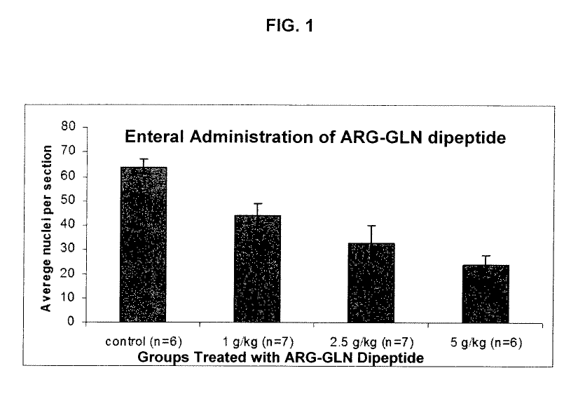

Results of enteral administration of the arginine-glutamine dipeptide are shown in FIG. 1, which is a bar chart showing the average nuclei per section in the eyes of neonatal mice exposed to hyperoxia as a function of the dosage of arginine-glutamine dipeptide, with dosage levels of: zero (control); 1 g/kg·day; 2.5 g/kg·day; and 5 g/kg·day. The greater number of nuclei corresponds to greater retinal vascular proliferation and therefore retinopathy. A lower number of nuclei, therefore, indicates a more favorable outcome. In FIG. 1, it is seen that the average number of nuclei per section was decreased at all levels of administration of the dipeptide as compared with subjects receiving only the control. A recognizable dose/response was shown for dosages of the dipeptide between 1 g/kg·day and 5 g/kg·day, and the highest level of effectiveness was shown at 5 g/kg·day.

### Example 2

This example illustrates the efficacy of enteral administration of arginyl-glutamine dipeptide in protecting against intestinal and brain injury induced by hyperoxia in a mouse model.

The C57BL6/J strain of timed pregnant mice was obtained from Jackson Laboratories (Bar Harbor, Me.). The mice were housed in the University of Florida Health Science Center Animal Care facilities.

In the neonatal mouse model of oxygen-induced retinopathy, 7-day old mice are placed with their nursing dams in a 75% oxygen atmosphere for 5 days. Mouse pups receive twice a day gavage feedings of arginine-glutamine dipeptide or control solution (20 μL) starting on postnatal day 12 (P12) and continuing through postnatal day 17 (P17). In one experiment, different dosages of arginine-glutamine dipeptide (1.0, 2.5 and 5 g/kg·day as a hydrochloride salt, Bachem, Babendorf, Switzerland) are tested. In a second experiment, arginine-glutamine dipeptide (5 g/kg day) is given and a normoxia group is used as a control.

After the fifth day following return to normoxia (P17), the animals are euthanized by injection of a lethal dose of a combination of ketamine (70 mg/kg body weight) and xylazine (15 mg/kg body weight) followed by cervical dislocation.

Tissue total myeloperoxidase (MPO) activity, a measure of neutrophil accumulation and a marker of tissue injury, is determined by a standard enzymatic procedure. Briefly, intestine samples are homogenized on ice in 0.01 M KH₂PO₄ buffer. After centrifugation at 10,000 g for 20 minutes at 4° C., the pellets are resuspended by sonication in cetyltrimethylammonium bromide buffer (13.7 mM CTAB, 50 mM KH₂PO₄, and 50 mM acetic acid, pH 6.0). The supernatant is kept for analysis. The suspension was centrifuged again at 10,000 g for 15 minutes, and the supernatant is then incubated in a 60° C. water bath for 2 hours. The MPO concentration of the supernatant is measured by the H₂O₂-dependent oxidation of tetramethylbenzidine. The absorbance is determined at 650 nm and compared with a linear standard curve. The amount of protein is measured using the BioRad Dc Protein Assay (Carlsbad, Calif.).

Bcl-2 levels are determined using standard immunoblotting techniques. The brain is homogenized with a Polytron homogenizer, and the homogenates are stored in aliquots at −80° C. Gel electrophoresis is performed using the BioRad electrophoresis system (Carlsbad, Calif.). Intestinal protein samples and Kaleidoscope pre-stained protein standards (BioRad, Carlsbad, Calif.) are loaded on a 12.5% (w·v) acrylamide Criterion pre-cast gel (BioRad, Carlsbad, Calif.) and electrophoresed at 100 volts for about 2 hours. Protein bands are electroblotted onto a polyvinyldifluoride membrane (PVDF) (Millipore Corporation, Bedford, Mass.) at 100 volts for 1 hour. Visualization of the protein bands is performed by staining the membrane with amido black. PVDF membranes are blocked for 1 hour in 5% (w·v) non-fat dried milk (NFDM) in Tris-buffered saline containing 0.1% Tween-20 (TBST) (Fisher Scientific, Atlanta, Ga.). Using fresh NFDM-TBST, the membranes are incubated with rabbit anti-Bcl-2 antibodies (Santa Cruz, Calif.), at 1:1000 dilution overnight at 4° C. with gentle rocking. The membranes are washed with multiple changes of TBST and subsequently incubated with a goat-anti-rabbit horseradish peroxidase-conjugated secondary antibody. ECL-Plus™, a chemiluminescent substrate (General Electric Healthcare, Piscataway, N.J.), is then applied to the membrane and incubated for 5 minutes at room temperature. Protein bands are visualized by exposure of membrane to X-OMAT scientific imaging film (Eastman-Kodak Corporation, Rochester, N.Y.), followed by development using a Kodak M35A X-OMAT processor (Kodak Diagnostic Imaging Inc., Rochester, N.Y.). Protein bands are quantified by densitometry.

Intestinal injury is evaluated using histological techniques. A portion of intestine from the ileum is fixed in 10% (w·v) neutral buffered formalin for 24 hours for light microscopy. The intestine is paraffin embedded, cut into 4 μm sections, mounted on glass slides, and stained with hematoxylin and eosin according to standard procedures. Histological analyses are performed using a damage scoring system from 0 to 4+ (0=no damage, 4=severe damage with mucosal sloughing) to determine the severity of the intestinal injury.

Caspase-3 activity is determined using a Caspase-3 Colorimetric Activity Assay Kit (Chemicon International, Inc., Temecula, Calif.). The brain is homogenized with a Polytron homogenizer, and the homogenates are stored in aliquots at −80° C.

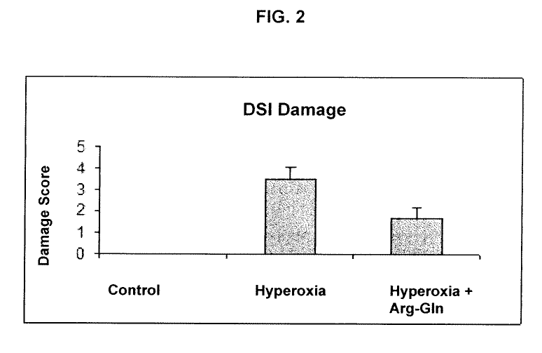

With respect to intestinal development, the results of the administration of the arginine-glutamine dipeptide are shown in FIG. 2, which is a bar chart showing the intestinal damage score under three different conditions: control; hyperoxic conditions; and hyperoxic conditions combined with arginine-glutamine dipeptide treatment. The damage score decreased when the subject was administered the arginine-glutamine dipeptide. The arginine-glutamine dipeptide protected against intestinal injury with an average damage score of 1.67. The average damage score of intestines exposed to hyperoxic conditions was 3.5.

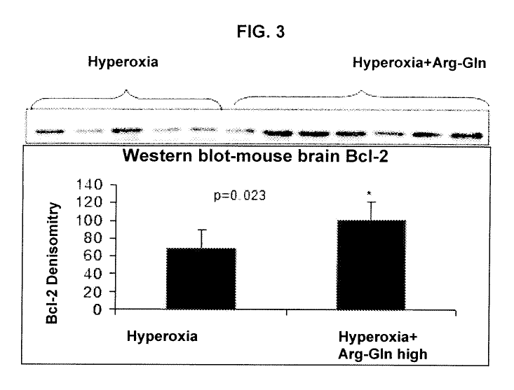

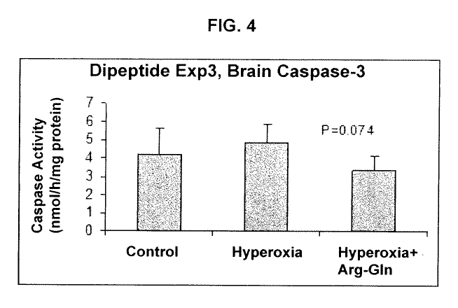

Regarding nervous system development, the results of the administration of the arginine-glutamine dipeptide are shown in FIGS. 3 and 4. FIG. 3 is a bar chart showing an increased Bcl-2 expression of 33% in a mouse brain treated with arginine-glutamine dipeptide and exposed to hyperoxic conditions. FIG. 4 shows a 32% reduction in brain caspase-3 activity compared to hyperoxic mice.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the disclosure are achieved and other advantageous results obtained.

As various changes could be made in the above methods and compositions by those of ordinary skill in the art without departing from the scope of the disclosure, it is intended that all matter contained in the above description and shown in the accompanying drawings shall be interpreted as illustrative and not in a limiting sense. In addition it should be understood that embodiments of the various embodiments may be interchanged both in whole and in part.

What is claimed is:

1. A method for supporting retinal, intestinal, or nervous system development in a neonate, the method comprising enterally administering arginine-glutamine dipeptide to a neonate to provide an amount of arginine-glutamine dipeptide that is effective to support retinal, intestinal, or nervous system development.

2. The method according to claim 1, wherein the total amount of arginine-glutamine dipeptide administered is from about 100 mg/kg·day to about 1000 mg/kg·day.

3. The method according to claim 1, wherein the neonate is in need of support of one or more of intestinal development, nervous system development, and retinal development.

4. The method according to claim 1, wherein the neonate is a premature infant.

5. The method according to claim 1, wherein the neonate is a postmature infant

6. The method according to claim 1, wherein the neonate is a full-term newborn infant.

7. The method according to claim 1, wherein enterally administering arginine-glutamine dipeptide comprises administering liquid infant formula containing arginine-glutamine dipeptide to the neonate.

8. The method according to claim 7, wherein the liquid infant formula is nutritionally complete.

9. The method according to claim 1, wherein enterally administering arginine-glutamine dipeptide comprises administering a nutritional supplement containing arginine-glutamine dipeptide to the neonate.

10. The method according to claim 1, wherein the neonate is in need of hyperoxic treatment.

11. An infant formula comprising arginine-glutamine dipeptide in an amount that is effective to support one or more of retinal, intestinal, and nervous system development in a neonate.

12. The infant formula according to claim 11, wherein the infant formula comprises the arginine-glutamine dipeptide in a total amount to provide the arginine-glutamine dipeptide to a neonate in an amount of from about 100 mg/kg·day to about 1000 mg/kg·day.

13. The infant formula according to claim 12, wherein the formula is a liquid in which the concentration of the arginine-glutamine dipeptide is from about 0.4 g/L to about 5 g/L.

14. The infant formula according to claim 12, wherein the infant formula is nutritionally complete and further comprises:
(a) a fat source;
(b) a carbohydrate source;
(c) a lipid source;
(d) a stabilizer; and
(e) a protein source.

15. A human milk fortifier comprising arginine-glutamine dipeptide in an amount that is effective to support one or more of retinal, intestinal, and nervous system development in a neonate.

16. The human milk fortifier according to claim 15, wherein the human milk fortifier comprises the arginine-glutamine dipeptide in a total amount to provide the arginine-glutamine dipeptide to a neonate in an amount of from about 100 mg/kg·day to about 1000 mg/kg·day.

17. The human milk fortifier according to claim 16, wherein the concentration of the arginine-glutamine dipeptide will result in a total concentration of arginine-glutamine dipeptide of from about 0.4 g/L to about 5 g/L in human milk.

18. An infant nutritional supplement comprising arginine-glutamine dipeptide in an amount that is effective to support one or more of retinal, intestinal, and nervous system development in a neonate.

19. The infant nutritional supplement according to claim 18, wherein the infant nutritional supplement comprises the arginine-glutamine dipeptide in a total amount to provide the arginine-glutamine dipeptide to a neonate in an amount of from about 100 mg/kg·day to about 1000 mg/kg·day.

20. The infant nutritional supplement according to claim 19, wherein the concentration of the arginine-glutamine dipeptide will result in a total concentration of arginine-glutamine dipeptide of from about 0.4 g/L to about 5 g/L in the infant nutritional supplement.
